SEQUENCE LISTING (13 sequences)

SEQ ID NO: 1 - protein, 854 aa, Hermetia illucens
MSRLLQKLCHLIQFSAFVILTTCVLGNLSAWQENVRPKLYVELGSEDVLRFAGNETAIDHFKLVLRDGNSLLVGARNVVFNLSIHDLTEQQRLLWSSPEDDVKMCVMKGKDEEACQNYIRTMVITAPGRLLICGTNSFRPKCHYYQINANNYSLEAEKSGQVVCPYDPKHNSTAVFADNELYSGTVADFSGSDPIIYREPLQTEQYDSLSLNAPNFVGSFTQGEFVYFLFRETAVEYINCGKAVYSRIARVCKWDKGGPNRFRNRWTSFLKSRLNCSMPGDFPFYFNEIQAASSLVEGSYGHTTSRLIYATFTTPVNAIPGSAVCAFSLQDISDTFEGNFKEQSGINSNWLPVNSAKVPDPRPGSCHNDSRTLPELTLNFIKTHSLMDENVPAFFGQPILVRTSTMYKFTYIAVDPQIKTPGGKTYDVIFVGTDHGKVIKTVNAESADSNKKVNSVVIEELDVLTKNEPIRNLEIVRTTTYDQAKDGNFDDGKLVIVTDSEVVAIKLHRCNSDKITSCSECVALQDPYCAWDKIAGKCRSHGAPRWLEENYFYQNVQTGQHAACPSGKMSKDANVGEQKGYRDMDLMDSHRNKDASGGEMINIMQDKNFDGSGPQISADVINAQYSVETLVMAVLAGAIFALLVGFITGYFCGRRCHKDEDDNLPYPDTEYEYFEQRQNVNRIQAEPKLLPQVEEVTYAEPVLLPQASSQNKIQHSPKNTLRKPMHHHGANSETLFQFQPDGYNTRDTYRGRDNFGTLRSHQVLGDNYRRGDGFSTTRSVKKGLNTNTRNRSLGRMRRQPPRHGIVTQHRSDSPQNSSSGSSPVMSHSSSSPAPPSSSPSPQESPKNCSYIYRD

SEQ ID NO: 2 - DNA, 2565 bp, Hermetia illucens
  source 1..2565: mol_type = other DNA
atgtctcggttattacaaaaattatgtcatttgatccaatttagtgcattcgtaattctaacaacatgtgtcctcggaaatttaagtgcctggcaagaaaacgtacgaccgaaactctatgttgaattgggttcagaagatgtgttacgattcgcgggaaatgaaacggccattgatcatttcaaattggtattacgagatggaaacagtttactggtgggagctaggaacgtagtcttcaacctgagcatacatgacttgactgaacaacaacgactcctgtggtcgtcgcctgaagatgatgtgaaaatgtgcgttatgaaaggaaaagacgaggaagcatgccagaattacatccgcaccatggtgataacggcccccggacgcttgttaatatgcgggacaaattcatttcgtcctaaatgtcattattatcaaattaatgcaaacaactactcccttgaggcagagaagagtggacaagttgtctgcccgtacgacccgaaacataattcgactgcggtttttgcagacaatgaattatattcgggaacggttgccgactttagtggaagtgatcctataatttatcgagagcctttgcagacggaacaatatgatagtctaagcttaaatgctccgaatttcgtgggatcattcacacagggcgaatttgtgtatttcctatttcgagaaacagctgttgaatacataaattgcggaaaggccgtatattcacgaatagcaagagtttgtaaatgggataagggaggaccgaatcgatttcgaaaccggtggacgtcctttttaaagtctcgattaaattgctccatgcccggggatttcccgttctacttcaatgaaattcaagcggcaagtagccttgttgaaggttcctacggtcatacaacttctcggctgatttatgcaacgttcacgacgccggtaaacgctatcccaggttcagctgtttgtgcattctcgttgcaggacatatcggatacatttgagggaaatttcaaagagcaaagtggtatcaactctaattggcttccagtaaatagtgcaaaggttccggatccacgacccggctcatgccacaatgactctcgcactcttccagaacttactttaaattttattaaaactcattcactaatggatgaaaatgtgccagcatttttcggtcaaccgatattggttaggactagcactatgtataaatttacgtatatagctgttgatccgcaaataaagacgcccggcggtaaaacgtatgatgttattttcgtcggaacagatcacggtaaagtgataaaaacagtaaacgcagaatcagccgattcaaataagaaagtaaattcggttgtaatcgaagaacttgacgtactaacgaaaaatgaaccaatacgaaatttggaaattgtacgaactacgacgtatgatcaagcaaaagacggaaactttgatgatggcaagcttgtgatcgtcacagatagtgaagttgtagcgattaagttgcatcgatgtaatagtgataaaattacaagttgcagcgagtgtgtagcacttcaagatccttattgcgcctgggataaaattgctgggaaatgtcgttcacatggtgcaccgaggtggcttgaggaaaattacttctatcagaatgttcagacggggcagcatgcagcatgtccgtcagggaaaatgtccaaagatgctaatgtgggggaacagaagggatatcgtgatatggatttaatggattcacatagaaataaggatgctagtggtggagaaatgataaatattatgcaagataaaaattttgatggcagtggaccacaaatctctgccgatgtcataaacgcacaatactcagttgaaacattagtgatggctgtattagccggagcaatatttgccctacttgtgggcttcatcacaggctacttttgtggtagacgttgccacaaagacgaagatgataacttaccatatccagacacggaatatgaatatttcgaacaacgacaaaatgttaaccgtattcaagctgaaccgaaacttcttccgcaagtcgaagaagtgacatatgctgaaccagttcttctacctcaagcatctagtcaaaacaaaatacaacattcgccaaaaaatacattaagaaaaccaatgcaccatcacggagccaattccgaaacgctcttccaatttcaacccgatggttataatactcgtgacacatatcgtggacgtgataatttcggtacattaaggtctcatcaggtattgggtgacaattaccgacgcggagatggattttcgacaacacgcagcgtgaagaagggtctaaacacaaacacaagaaatagaagtcttggccgaatgaggaggcagccgccccggcatggtattgtaactcaacatcgttccgatagtccacaaaattcaagttcaggatcatcgcctgttatgtcacatagtagttctagtcctgcgccaccatcaagtagtccgagtccacaagagagcccgaaaaactgtagttatatataccgcgactga

SEQ ID NO: 3 - DNA, 297 bp, synthetic construct
  source 1..297: mol_type = other DNA
ttgtgtttgtaagcgaaaaacatcatttcattaatgcattctctgaaatagttcaattttccattttgcaggaagcatgccagaattacatccgggaacggttgccgactttagtggaagtgatcctataatttatcgagagcctttgcagacggaacaatatgatagtctaagcttaaatggtgggtatactctctcatttgtgtatataaaaggactatgtggacgctaatcaatattctccataaggaattgggcccactgtctcttatacacatctgacgctgccgacgagac

SEQ ID NO: 4 - DNA, 22 bp, synthetic construct
  source 1..22: mol_type = other DNA
gaggaggccaactaacagttcc

SEQ ID NO: 5 - DNA, 21 bp, synthetic construct
  source 1..21: mol_type = other DNA
tgggcccaattccttatggag

SEQ ID NO: 6 - DNA, 23 bp, synthetic construct
  source 1..23: mol_type = other DNA
gcctcaagggagtagttgtttgc

SEQ ID NO: 7 - DNA, 22 bp, synthetic construct
  source 1..22: mol_type = other DNA
ctggctgtgcgctcatatctag

SEQ ID NO: 8 - DNA, 75 bp, synthetic construct
  source 1..75: mol_type = other DNA
tagtgcattcgtaattctaacaacatgtgtcctcggaatttaagtgcctggcaagaaaacgtacgaccgaaactc

SEQ ID NO: 9 - DNA, 66 bp, synthetic construct
  source 1..66: mol_type = other DNA
tagtgcattcgtaattctaacaacatgtgtttaagtgcctggcaagaaaacgtacgaccgaaactc

SEQ ID NO: 10 - DNA, 20 bp, synthetic construct
  source 1..20: mol_type = other DNA
ccagaattacatccgcacca

SEQ ID NO: 11 - DNA, 20 bp, synthetic construct
  source 1..20: mol_type = other DNA
tcagacaatgaattatattc

SEQ ID NO: 12 - DNA, 20 bp, synthetic construct
  source 1..20: mol_type = other DNA
gccaggcacttaaatttccg

SEQ ID NO: 13 - DNA, 20 bp, synthetic construct
  source 1..20: mol_type = other DNA
tgtggactcggactacttga

(19) United States
(12) Patent Application Publication
(10) Pub. No.: US 2026/0041074 A1
(43) Pub. Date: Feb. 12, 2026

(54) HIGH-WEIGHT BLACK SOLDIER FLY LARVAE, METHODS OF PRODUCING SAME AND USE THEREOF

(71) Applicant: FREEZEM CRYOGENICS LTD., Nachshonim (IL)

(72) Inventors: Inbar Nevo Yassaf, Givattaim (IL); Anna Goren, Rehovot (IL); Idan Alyagor, Pardesia (IL)

(21) Appl. No.: 19/148,040

(22) PCT Filed: Feb. 29, 2024
(86) PCT No.: PCT/IL2024/050231

§ 371 Date: Jul. 15, 2025

(30) Foreign Application Priority Data
Mar. 2, 2023 (IL) 301082

(51) Int. Cl. A01K 67/68; A23J 3/04; A23K 10/20; A23L 13/10; B09B 3/60; C12N 9/22; C12N 15/11; C12N 15/90

(52) U.S. Cl. CPC A01K 67/68; A23J 3/04; A23K 10/20; A23L 13/10; B09B 3/60; C12N 9/226; C12N 15/11; C12N 15/902; A01K 2217/056; A01K 2217/075; A01K 2227/706; A01K 2267/00; C12N 2310/20

(57) ABSTRACT

The present invention relates to larvae of black soldier fly having high weight, to methods of producing same and to use of the high weight larvae in various applications including as a food or feed, as a source for food grade ingredients including proteins, chitin, and fat, and for conversion of waste into biomass.

### FIELD OF THE INVENTION

The present invention relates to larvae of black soldier fly having high weight, to methods of producing same and to use of the high weight larvae in various applications including as a food or feed, as a source for food grade ingredients including proteins and fat, and for conversion of waste into biomass.

### BACKGROUND OF THE INVENTION

The black soldier fly (BSF), Hermetia illucens (Diptera: Stratiomyidae), is a true fly of the family Stratiomyidae. Black soldier fly (BSF) larvae are feeding on an immense variety of organic material, and have been reported as one of the most efficient insects in converting organic waste into biomass. The BSF larvae can thus be used in waste management, and, since they are edible, as a feed for various farm animals as well as a food source.

Korean Application No. KR20210157505 relates to dog feed, using BSFs and an apparatus and a method for manufacturing the same. The system comprises a fermenter in which microbial fermentation for a predetermined time is performed on a BSFs-containing mixture; and an odor reducer capturing and burning the odor generated during the drying or fermentation of the mixture in the fermenter.

Chinese Application No. CN109006699 discloses an ecological breeding method for treatment of pig manure by employing black soldier fly larvae. The method comprises the steps of: larva processing, pig manure water content processing, and larva inoculation and pig manure treatment. In the process, feces are mixed with four days old larvae. The manure treatment process is simple, controllable, easy for application by the farmers and reduces the treatment cost.

Chinese Application No. CN107549126 discloses use of BSF larvae for waste management, particularly for treating kitchen-based garbage by growing BSF-larvae on kitchen organic waste, thereby converting the organic waste into biomass.

International PCT Application Publication No. WO 2020/234884, for example, discloses methods for modifying oil extracted from BSF larvae. Applications of the modified BSF larvae oil include dermal and/or oral applications, topical therapy, as well as applications to medical equipment and industrial applications.

The remarkable waste bioconversion ability of BSF larvae makes it a promising species to be mass-produced globally. Most of the food consumption and biomass gaining are achieved during the larval stage of the fly life cycle. Therefore, the extension of the larval stage will improve larval performance.

In insects, metamorphosis and sexual maturation requires changes in hormonal secretory activities of the steroidogenic prothoracic gland (PG). The commitment to metamorphosis depends robustly on nutrients available during the larvae growth and to the larvae body weight. Fed larvae must reach a “critical weight” in order to start maturation. After a critical weight is attained, a small release of the steroid prohormone ecdysone from the PG is set in motion. Few days later, a pulse of ecdysone triggers end of feeding, initiation of wandering, and entering the pupal stage. To this end little is known about how body fat is sensed in order to trigger the release of ecdysone.

Zhan et al. (Zhan S et al., 2020. Cell Research 30(1):50-60. doi:10.1038/s41422-019-0252-6) used a CRISPR/Cas9-based gene editing approach to explore BSF phenotypes. Using this approach, they have identified a prothoracicotropic hormone (PTTH) gene mutant having an enhanced feeding capacity phenotype. The PTTH gene is responsible for the initiation of a signaling cascade that culminates in the biosynthesis and release of ecdysone and by that contributes to metamorphosis. However, metamorphosis in the PTTH mutant BSF larvae was significantly delayed (from 4-5 days to more than 85 days) making it not relevant for industrial applications.

Juarez-Carreno et al. (Juarez-Carreno S et al., 2021. Cell Reports 37:109830; doi.org/10.1016/j.celrep.2021.109830) conducted an RNAi screen in order to identify genes, silencing of which resulted in failure to initiate sexual maturation. Using this approach, they found that signaling and sensing fat sufficiency for sexual maturation commitment requires the lipid carrier apolipophorin (apolpp) in fat cells and semaphorin-1a (Sema1a) in the neuroendocrine prothoracic gland (PG). RNAi silencing of Sema1a in the PG gland resulted in larvae that failed to maturate and continue eating and gaining weight until death, failing to metamorphose. Activation of PTTH/Ras pathways in the PG did not rescue the Sema1a-defficiency phenotype.

There is a growing need for, and it would be highly advantageous to have, high quality BSF larvae that maintain the BSF natural life cycle for the various uses of these larvae.

### SUMMARY OF THE INVENTION

The present invention answers the above-defined needs, providing high-weight black soldier fly (BSF) larvae that maintain their capability to metamorphose and subsequently emerge adult flies. The high-weight BSF larvae of the invention can be used in its whole form in animal feed, in meal form in food, as a source for food grade proteins, fat, vitamins and the like, as a source for industrial ingredient, e.g., chitin, and in waste management, wherein the high-weight of the larvae improves all the applications. The capability of the mutant larvae to complete the life-cycle is a pre-requisite for its use in a large-scale commercial rearing.

The present invention is based in part on the unexpected finding that BSF larvae genetically modified to have within its genome at least one mutation within the gene encoding axon guidance protein Semaphorin1a (Sema1a), were late to metamorphose into the pupal stage and were significantly larger at the end of the larval stage compared to a corresponding, not-genetically modified wild-type larvae, while showing no or negligible deleterious effects associated with the modification and maintaining their capability to transform into pupa and mature to adult fly.

The present invention further discloses edible compositions comprising the high weight larvae of the invention and use thereof as a feed/food. The present invention also relates to the use of the high-weight larvae as a source for nutritional components including, but not limited to, proteins and fat. The present invention also relates to the use of the high-weight larvae as a bioconversion tool for waste management applications.

According to certain aspects, the present invention provides a genetically modified black soldier fly larva (BSFL) having reduced expression and/or activity of Semaphorin1a (Sema1a) protein or a homolog thereof, wherein the weight at the end of the 5^{th} larval stage of the genetically modified larva is higher than the weight of a corresponding, unmodified BSFL grown under similar conditions and being at the same larval stage.

According to certain embodiments, the genetically modified BSFL is capable to metamorphose into pupae. According to certain embodiments, the genetically modified BSFL is capable to metamorphose into pupae and then into an adult fly. According to these embodiments, the fly is fertile. According to certain embodiments, the adult fly matured from the genetically modified larva has an increased weight compared to the weight of an adult fly matured from a corresponding unmodified BSFL grown under similar conditions.

According to certain embodiments, the genetically modified BSFL metamorphoses at least 1 day, at least 2 days, at least 3 days, at least 4 days or at least 5 days after the corresponding unmodified BSFL grown under similar conditions metamorphoses.

According to certain embodiments, the weight gain of the genetically modified BSFL is similar to the weight gain of the corresponding unmodified BSFL throughout the linear growth period.

As used herein, the term “linear growth period” of a BSF larva refers to a period of from about 4 to 5 days after hatching to about 10 to 14 days after hatching for wild type larva and 4 to 5 days after hatching to about 10 to 20 days after hatching under regular growth conditions for the genetically modified larva of the invention.

As used herein, the term “regular growth conditions” refers to growth at 27-30° C., 55-70% humidity, 50,000 larvae per square meter.

According to certain embodiments, the feed conversion ratio (FCR) of the genetically modified BSFL is lower compared to the FCR of the corresponding unmodified BSFL. According to some embodiments, the FCR is lower throughput the BSFL growth period. According to some embodiments, the FCR is lower at certain stages of the BSFL growth. According to certain embodiments, the FCR value of the genetically modified BSFL is improved by from about 10% to about 50% compared to the FCR of the corresponding unmodified BSFL. According to certain exemplary embodiments, the FCR is improved by from about 15% to about 25%.

According to certain embodiments, the Sema1a protein or homolog thereof comprises an amino acid sequence having at least 85% identity to the amino acid sequence set forth in SEQ ID NO:1.

According to certain exemplary embodiments, the Sema1a protein comprises the amino acid sequence set forth in SEQ ID NO:1.

According to certain embodiments, the Sema1a protein is encoded by a Sema1a gene or a homolog thereof having at least 85% identity to the nucleic acid sequence set forth between position 159,092,514 and position 159,592,196 within the nucleic acid sequence set forth in NCBI Reference Sequence NC_051851.1. According to these embodiments, the nucleic acid sequence of the Sema1a-protein encoding genomic sequence comprises 499,683 base pairs (bps).

According to certain embodiments, the Sema1a gene comprises the nucleic acid sequence set forth in NCBI Reference Sequence NC_051851.1 between position 159,092,514 and 159,592,196.

According to certain embodiment, the Sema1a protein is encoded by a nucleic acid sequence having at least 85% identity to the nucleic acid sequence set forth in SEQ ID NO: 2.

According to some embodiments, the Sema1a protein is encoded by the nucleic acid sequence set forth in SEQ ID NO:2.

According to certain embodiments, the genetically modified BSFL comprises within its genome at least one mutant allele of the Sema1a gene or of the homolog thereof.

According to certain embodiments, the mutant allele of Sema1a or of the homolog thereof comprises at least one mutation. According to certain embodiments, the mutant allele of Sema1a is designated herein Sema1a^{mut}.

According to certain embodiments, the Sema1a^{mut} allele or the homolog thereof comprises a single mutation. According to certain embodiments, the Sema1a^{mut} allele or the homolog thereof comprises a plurality of mutations.

According to certain embodiments, the Sema1a^{mut} allele or homolog thereof confers a reduced function or a loss of function of the encoded Sema1A protein.

Any mutation(s) can be inserted into the polynucleotide encoding Sema1a or homolog thereof, including deletions, insertions, insertion-deletion mutations (indels), site specific mutations including nucleotide substitution and the like, as long as the mutation(s) result in down-regulation of the gene expression and in reduced expression of the Sema1a protein and/or in the production of less-functional or non-functional protein or homologs thereof.

According to certain embodiments, the Sema1a^{mut} allele or the homolog thereof comprises mutation in its Sema domain. According to certain embodiments, the Sema1a^{mut} allele or the homolog thereof comprises mutation outside its Sema domain.

According to certain exemplary embodiments, the at least one mutation within the at least one allele is a deletion mutation. According to further exemplary embodiments, the at least one deletion comprises an exon or a part thereof. According to further certain exemplary embodiments, the at least one mutation within the at least one allele results is a deletion mutation leading to a premature stop codon.

According to certain exemplary embodiments, the at least one mutation in the Sema1a^{mut} allele is a deletion within the genomic sequence encoding the Sema1a protein. According to certain exemplary embodiments, the deletion is of 50987 nucleotides from position 159,121,513 to position 159,172,499 on the nucleic acid sequence set forth in NCBI Reference Sequence: NC_051851.1.

According to certain exemplary embodiments, the Sema1a^{mut} allele comprises the nucleic acid sequence set forth in SEQ ID NO:3.

According to certain embodiments, the at least one mutation in the Sema1a^{mut} allele is a deletion within the coding sequence of the Sema1a gene (SEQ ID NO:2). According to further certain exemplary embodiments, the at least one mutation is a deletion of the nucleotide A (Adenine) at position 80 of the nucleic acid sequence set forth in SEQ ID NO: 2. According to these embodiments, the Sema1a^{mut} allele comprises the nucleic acid sequence set forth in SEQ ID NO:8.

According to yet additional certain exemplary embodiments, the at least one mutation is a deletion of the 10 nucleotides (CCTCGGAAAT) at position 72 to position 81 of the nucleic acid sequence set forth in SEQ ID NO:2. According to these embodiments, the Sema1a^{mut} allele comprises the nucleic acid sequence set forth in SEQ ID NO: 9.

According to some embodiments, the genetically modified BSFL is heterozygous to the Sema1a^{mut} allele.

According to some embodiments, the genetically modified BSFL is homozygous to the Sema1a^{mut} allele.

Any method for mutagenesis as is known in the art and suitable to use with black soldier fly (BSF) can be used according to the teachings of the present invention. Mutagenesis methods include, but are not limited to, chemical mutagenesis, radio-mutagenesis and site directed mutagenesis, for example using genome editing techniques.

According to certain embodiments, the mutation is a site-specific mutation generated by a method selected from the group consisting of, but not limited to, site-directed plasmid mutagenesis and gene-editing method using artificially engineered nucleases.

According to certain embodiments, the artificially engineered nucleases are selected from the group consisting of meganucleases, Zinc finger nucleases (ZFNs), transcription-activator like effector nucleases (TALENs), and CRISPR/Cas, CRISPR/Cas homologous and CRISPR/Cas modified systems.

Generation of site-specific mutations, particularly using gene-editing systems, has the advantage of designing mutagenesis tools that do not have off-target effects.

Thus, according to certain exemplary embodiments, the genetically modified BSFL of the present invention having higher weight compared to unmodified BSFL are obtained by generating a mutation within at least one allele of Sema1a using CRISPR/Cas system.

According to certain embodiments, the weight of the genetically modified BSFL is higher by at least 10%, at least 20%, at least 30%, at least 40%, at least 50% or more compared to the weight of the corresponding unmodified BSFL.

According to certain embodiments, the protein content of the genetically modified BSFL comprise at least 10%, at least 20%, at least 30%, at least 40%, at least 50% w/w protein, or more, out of the total weight of said BSFL based on a dry weight.

According to certain exemplary embodiments, the genetically modified BSFL comprise from about 25% to about 50% w/w protein out of the total weight of said BSFL based on a dry weight.

According to certain embodiments, the genetically modified BSFL comprise at least 10%, at least 20%, at least 30%, at least 40%, at least 50% or more w/w fat out of the total weight of said BSFL based on a dry weight.

According to certain exemplary embodiments, the genetically modified BSFL comprise from about 20% to about 50% w/w fat out of the total weight of said BSFL based on a dry weight.

According to certain additional aspects, the present invention provides an edible composition comprising a plurality of the genetically modified BSFL of the invention and/or parts thereof. According to certain embodiments, the edible composition further comprising at least one food-grade excipient or carrier.

According to certain embodiments, the edible composition comprises at least one additional nutritional component selected from the group consisting of at least one food-grade protein and/or amino acids, at least one food grade carbohydrate, at least one food grade fatty acid and any combination thereof. Each possibility represents a separate embodiment of the present invention.

According to certain embodiments, the edible composition comprises the genetically modified BSFL or parts thereof is in a form selected from the group consisting of a living form, a dried form, and a combination thereof. Each possibility represents a separate embodiment of the present invention.

According to certain embodiments, the dried genetically modified BSFL is in a form selected from the group consisting of whole larva and a larva meal.

According to certain embodiments, the dried genetically modified BSFL is defatted.

According to certain embodiments, the edible composition is for feeding a non-human animal. According to certain embodiments, the non-human animal is selected from the group consisting of a land animal and an aquatic animal. According to certain embodiments, the aquatic animal is selected from the group consisting of fish and crustacean. According to certain embodiments, the land animal is selected from the group consisting of avian, reptile, and mammal farm animal. According to certain embodiments, the animal is an insect.

According to certain embodiments, the edible composition is a food for humans. According to these embodiments, the plurality of genetically modified BSFL is in a dried form.

According to further aspects, the present invention provides a method for producing a high-weight BSFL, the method comprising reducing the expression and/or activity of Sema1a protein within at least one cell of the BSFL. According to certain embodiments, the method results is a BSFL having higher weight compared to the weight of a corresponding BSFL having unmodified expression and/or activity of Sema1a protein.

According to certain embodiments, the method comprises generating at least one mutation in at least one wild type allele of Sema1a to form Sema1a1^{mut} allele, wherein the at least one mutation confers a loss of function, a reduced function, or an abnormal function of the encoded Sema1a protein.

The wild type Sema1a protein, the gene encoding same, and methods of generating the at least one mutation are as described hereinabove.

According to certain exemplary embodiments, the at least one mutation is induced by genome editing using the CRISPR/Cas system.

Also encompassed herein are BSFL produced by the method of the invention and parts thereof.

According to yet additional aspects, the present invention provides a composition comprising at least one fraction derived from the genetically modified BSFL of the invention, wherein the at least one fraction is selected from the group consisting of protein fraction, fat fraction, vitamin and mineral fraction, chitin fraction, and any combination thereof.

According to certain embodiments, the mineral fraction is an aqueous fraction.

Any method as is known in the art for obtaining protein fraction, fat fraction or vitamin containing aqueous fraction, chitin fraction, as well as for obtaining isolated proteins and/or components thereof, fat and/or component thereof and/or vitamins and/or minerals can be used according to the teachings of the present invention.

According to yet additional aspects, the present invention provides a method of converting an organic waste to biomass, the method comprises providing a plurality of the BSFL of the invention with organic waste as the sole nutrient source.

According to certain embodiments, the organic waste is a household organic waste. According to certain additional or alternative embodiments, the organic waste is agricultural waste.

According to certain embodiments, the method further comprises a step of separating non-organic and/or toxic compound from the organic waste.

According to certain embodiments, the method results in a weight gain of at least 25%, at least 50%, at least 100%, at least 150%, at least 200%, at least 250% or more of the average initial weight of the plurality of genetically modified BSFL.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows the generation of Semaphorin1A (Sema1a) deletion mutation. FIG. 1A is a schematic representation of exons/introns of the Sema1a gene in Hermetia illucens. Exons are shown as boxes; introns are represented by narrow lines. Guide RNA (gRNA) locations are noted and Protospacer Adjacent Motif (PAM) sites are labeled in bold face. Deletion is represented by the double head arow. The total length of the deletion is 50987 bp and the distance between the gRNAs is 50961 bp. FIG. 1B shows the targeted locus in Sema1a gene and the deletion in the mutant line.

FIG. 2 shows mutations formed using the CRISPR-Cas9 system with the sgRNA sgD (SEQ ID NO:13).

FIG. 3 shows mutations formed using the CRISPR-Cas9 system with the sgRNA sgA (SEQ ID NO:12).

FIG. 4 is a schematic presentation of the deletion mutation within the Sema1a coding region and the resulted non-functional protein. FIG. 4A: Sema1a mRNA illustration. FIG. 4B: Mutation positions on SEQ ID NO:2. FIG. 4C: Schematic presentation of the premature stop codon within the Sema1a protein resulting from the mutations shown in FIG. 4B. Numbers and single letters represents the amino acid positions on SEQ ID NO:2. Three-letter groups represent the codon encoding each amino acid. * Denotes stop codon.

FIG. 5 demonstrates characteristics of Sema1a^{mut} phenotype. FIG. 5A: Shown is the population of mutant larvae of Sema1a and control at day 16 after hatching from the egg. FIG. 5B: Representative CRISPR mutant larvae of the Sema1a gene and WT control larva at day 14 after hatching from the egg. FIG. 5C: Average larval weight of WT control and Sema1a mutants (n=30; mean values±SEM). FIG. 5D: Time course of larval weight in control and Sema1a mutants during day 6-14 after hatching from the egg. FIG. 5E: Shown is a representative CRISPR mutant fly of the Sema1a gene and WT control (upper panel). Body parts of Sema1a^{mut} and WT flies wing, metathoracic right leg, and head (lower panel). FIG. 5F: Weight of CRISPR mutant fly of the Sema1a gene and WT control.

FIG. 6 shows weight gain of “Titan” mutant larvae (larvae comprising a Sema1a^{mut} comprising SEQ ID NO:9) and wild type (WT) larvae up to blackening.

FIG. 7 is a representative picture of wild type (WT) and mutant larva (larvae comprising a Sema1a^{mut} allele comprising SEQ ID NO:9) up to the blackening points. Day number − days after hatching from the egg.

FIG. 8 shows percentages of larvae reaching blackening of (WT) and “Titan” mutant larva (larvae comprising a Sema1a^{mut} allele comprising SEQ ID NO:9).

### DETAILED DESCRIPTION

The present invention relates to the field of insect rearing and use, particularly to the production of black soldier fly (BSF) larvae having at least one mutation in the Semaphorin-encoding gene Sema1a or a homolog thereof, which reach high weight before metamorphosing into pupa, and mature to fertile, high weight adult fly. The genetically modified BSF larvae of the invention show improved feed conversion ratio (FCR) compared to corresponding unmodified BSF larvae grown under the same conditions, and protein, fat, and chitin profiles comparable to corresponding larvae expressing wild type Sema1a.

The genetically modified BSF Larvae of the invention can be utilized for all known and to be known uses of insect larvae, including as an animal feed, as an ingredient within animal feed or human food compositions, as a source for nutritional components including, inter alia, proteins, fats, minerals, and vitamins, as waste management agents, and in rearing adult BSF flies.

### Definitions

The singular forms “a”, “an”, and “the” include plural referents unless the context clearly dictates otherwise.

Unless the context clearly requires otherwise, throughout the specification, the words “comprise”, “comprising” and the like are to be construed in an inclusive sense as opposed to an exclusive or exhaustive sense; that is to say, in the sense of “including, but not limited to”.

The term “weight” when used with regard to larva weight refers to the wet weight of living larva at a certain day after laying/hatching.

As used herein, the term “about” is to be understood as within a range of normal tolerance in the art, for example within 2 standard deviations of the mean. About can be understood as within +10%, 9%, 8%, 7%, 6%, 5%, 4%, 3%, 2%, 1%, 0.5%, 0.1%, 0.05%, or 0.01% of the stated value. All values provided herein are understood to be modified by the term about.

As used herein, the term “unmodified BSF larva” refers to larva of BSF fly in which the expression of its endogenous Sema1a protein or its homolog has not been artificially modified. According to certain exemplary embodiments, the unmodified BSF larva expresses the wild type Sema1a protein or homolog thereof having an amino acid sequence at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more identical to the amino acid sequence set forth in SEQ ID NO:1.

The genomic sequence encoding the wild type BSF Sema1a protein comprises 499,683 bp located between positions 159,092,514 and position 159,592,196 of the nucleic acid sequence set forth in NCBI Reference Sequence NC_051851.1 (designated in Israel Patent Application No. 301082, being the priority of the present application as SEQ ID NO:2).

According to certain embodiments, the wild type Sema1a protein or the homolog thereof is encoded by a nucleic acid sequence at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more identical to the amino acid sequence set forth in SEQ ID NO:2 (cDNA encoding sequence).

It is to be explicitly understood that the “unmodified BSFL” can comprise other modifications, for example modified expression and/or activity of proteins other than Sema1a. According to certain embodiments, the term “corresponding” with regard to BSFL refers to larva of BSF flies of the same variety.

Homology (e.g., percent homology, sequence identity+sequence similarity) can be determined using any homology comparison software computing a pairwise sequence alignment.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage of sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. (Amino acid substitution matrices from protein blocks. Proc. Natl. Acad. Sci. U.S.A. 89(22), 10915-9, 1992).

Identity (e.g., percent homology) can be determined using any homology comparison software, including for example, the BlastN, BlastX or Blastp software of the National Center of Biotechnology Information (NCBI) such as by using default parameters.

According to some embodiments of the invention, the identity is a global identity, i.e., an identity over the entire amino acid or nucleic acid sequences of the invention and not over portions thereof.

According to some embodiments of the invention, the term “homology” or “homologous” refers to identity of two or more nucleic acid sequences; or identity of two or more amino acid sequences; or the identity of an amino acid sequence to one or more nucleic acid sequences.

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. It is to be explicitly understood that the terms “Sema1a allele” and Sema1a mutant allele” (“Sema1a^{mut}”) encompass the genomic sequence as well as the mRNA encoding the wild type or mutant Sema1a protein.

The terms “polynucleotide”, “polynucleotide sequence”, “nucleic acid sequence”, and “isolated polynucleotide” are used interchangeably herein. These terms encompass isolated nucleotide sequences and the like. A polynucleotide may be a polymer of RNA or DNA or hybrid thereof, that is single- or double-stranded, linear, or branched, and that optionally contains synthetic, non-natural or altered nucleotide bases. The terms also encompass RNA/DNA hybrids.

The term “plurality” as used herein refers to at least two. According to certain embodiments of the invention, a plurality of mutation within Sema1a^{mut} allele comprises two, three, four, five, six or more mutations. Each possibility represents a separate embodiment of the present invention.

According to certain aspects, the present invention provides a genetically modified black soldier fly larva (BSFL) having reduced expression and/or activity of Semaphorin1a (Sema1a) protein or a homolog thereof, wherein the weight at the end of the 5^{th} larval stage of the genetically modified BSFL is higher than the weight of a corresponding, unmodified BSFL grown under the same conditions and being at the same development stage.

Semaphorin is a family of glycoproteins, the members of which are regulatory molecules in the development of the nervous system and in axonal guidance. They also play important roles in other biological processes, such as angiogenesis, immune regulation, and respiration systems. Sema1a known mutant phenotypes mainly result from defects in the nervous system such as abnormal locomotor behavior, and abnormal neuroanatomy (e.g., Shen H C et al. 2017. PLOS Genetics 13(4):e1006751. doi.org/10.1371/journal.pgen.1006751; Hernandez-Fleming M et al., 2017, Cell Reports 18:174-184; Cafferty P et al. 2006. The Journal of Neuroscience, 26(15):3999-4003).

According to certain embodiments, the semaphorin protein according to the teachings of the present invention is Hermetia illucens semaphorin or a homolog thereof.

According to certain embodiments, the Hermetia illucens semaphorin comprises the amino acid sequence set forth in SEQ ID NO:1 (NCBI Reference Sequence: XP_037911809.1).

As used herein, the expression and/or activity of Sema1a protein is “reduced”, “inhibited”, “down regulated” or “knocked out” or “knocked down” if the level of the Sema1a encoding gene, the encoded protein or the protein measured activity is reduced by at least 20%, at least 30%, at least 40%, at least 50%, at least 60%, at least 65%, at least 70%, at least 75%, at least 80%, at least 85%, at least 90%, at least 91%, at least 92%, at least 93%, at least 94%, at least 95%, at least 96% at least 97%, at least 98%, at least 99%, or more compared to the level in a corresponding cells or BSF larva not genetically modified according to the teachings of the invention. It is to be explicitly understood that a “reduced activity” encompasses expression of an abnormal and/or modified protein leading to reduced normal function and/or no function or an abnormal function of the Sema1a protein.

According to certain embodiments, the reduced expression and/or activity of Sema1a protein does not negatively affect the larva maturation.

Growth of wild type BSF larvae typically comprises three phases, including a first phase of a slow growth rate (from hatching up to 4-5 days old larva, or a weight of about 5 mg), a second phase of fast linear growth (from 4-5 days up to 10-14 days, from about 5 mg to about 200 mg) and a third phase of growth arrest (after the fast linear growth), during which the larvae are blackening. The high-weight genetically modified BSFLs of the invention show a second phase of liner growth of 10-20 days, starting from an initial weight of about 5 mg and reaching a weight of about 350 mg or more at the end of the linear phase.

According to certain embodiments, the weight of the genetically modified BSFLs of the invention at the end of the liner phase is about 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 310 mg, 320 mg, 330 mg, 340, about 350 mg or more.

According to certain embodiments, the weight of the genetically modified BSFL is equal or higher compared to the weight of the corresponding unmodified BSFL throughout the growth period of the larva.

According to certain alternative embodiments, the weight of the genetically modified BSFL is equal or higher compared to the weight of the unmodified BSFL through the liner growth phase of the larva.

According to certain embodiments, the weight of the genetically modified BSFL of the invention is at least about 10%, at least about 15%, at least about 20%, at least about 25%, at least about 30%, at least about 35%, at least about 40%, at least about 45%, at least about 50%, at least about 55%, at least about 60%, at least about 65% or more higher compared to the weight of a corresponding unmodified BSFL grown under similar condition and being at the same day after egg laying.

According to certain exemplary embodiments, the weight of the genetically modified BSFL of the invention is from about 40% to about 60% higher compared to the weight of the corresponding unmodified BSFL.

Feed conversion ratio (FCR) is the conventional measure of production efficiency in terms of the conversion of feed consumed (input) to the desired output. FCR of BSFL is the weight of feed intake divided by weight gained by the larva.

According to certain embodiments, the feed conversion ratio (FCR) value of the genetically modified BSFL is lower compared to the FCR value of the corresponding unmodified BSFL. According to some embodiments, the FCR is lower throughout the BSFL growth period. According to some embodiments, the FCR is lower at certain stages of the BSFL growth. According to some embodiments, the FCR is lower at the linear growth phase.

According to certain embodiments, the FCR value of the genetically modified BSFL of the invention is about 10% to about 50% lower compared to the FCR value of corresponding unmodified BSFL. According to some embodiments, the FCR value of the genetically modified BSFL is about 15%, about 16%, about 17%, about 18%, about 19%, about 20%, about 21%, about 22%, about 23%, about 24%, or about 25% lower compared to the FCR value of corresponding unmodified BSFL.

According to certain embodiments, the entire growth period from hatching to the industrial harvesting stage of the genetically modified BSFL is shorter compared to the growth period of the corresponding unmodified larvae. According to certain embodiments, the entire growth period from egg laying to industrial harvesting stage of the genetically modified BSFL is at least one days shorter, at least 2 days shorter, at least 3 days shorter, at least 4 days shorter or at least 5 days shorter. Each possibility represents a separate embodiment of the present invention.

Industrial harvesting stage is typically taken as the stage in which a wild type larva reaches a weight of about 150-180 mg. It is to be explicitly understood that the genetically modified larvae of the present invention can further grow and reach a weight above 180 mg, typically about 250 mg to 300 mg, before entering the growth arrest phase and being harvested.

According to certain embodiments, the genetically modified BSFL is capable to metamorphose into pupae. According to certain embodiments, the genetically modified BSFL is capable to metamorphose into pupae and then into an adult fly. According to these embodiments, the fly is fertile. The capability of the genetically modified BSFL of the invention to complete the life cycle up to fertile fly is of significant importance in the commercial rearing of the larvae of the invention.

According to certain embodiments, the adult fly matured from the genetically modified larva has an increased weight compared to the weight of an adult fly matured from a corresponding unmodified BSFL grown under the same conditions.

According to certain embodiments, the genetically modified BSFL metamorphose at least one day, at least two days, at least three days, at least four days, at least 5 days, or at least 6 days after the corresponding unmodified larvae metamorphose. Each possibility represents a separate embodiment of the present invention.

According to certain embodiments, the weight of the fly matured from the genetically modified BSFL of the invention is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or more, higher compared to the weight of a fly matured from a corresponding unmodified BSFL, wherein the fly is grown under the same condition and being at the same day after hatching.

According to certain exemplary embodiments, the weight of the fly matured from the genetically modified BSFL of the invention is from about 20% to about 80% higher compared to the weight of the corresponding unmodified BSFL.

According to certain embodiments, the weight of the fly matured from the genetically modified BSFL of the invention is from about 30% to about 50% higher compared to the weight of the corresponding unmodified BSFL.

According to certain embodiments, the Sema1a protein or homolog thereof comprises an amino acid sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity, or being identical, to SEQ ID NO:1.

According to certain embodiments, the Sema1a protein or homolog thereof is encoded by a polynucleotide comprising a nucleic acid sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity, or being identical, to SEQ ID NO:2.

Any mutation(s) can be inserted into the polynucleotide encoding Sema1a protein or a homolog thereof, including deletions, insertions, insertion-deletion mutations (indels), site specific mutations including nucleotide substitution and the like, as long as the mutation(s) result in down-regulation of the gene expression or in the production of less-functional or non-functional protein.

Any method for mutagenesis as is known in the art can be used according to the teachings of the present invention including chemical mutagenesis, radio-mutagenesis and site directed mutagenesis, for example using genome editing techniques.

According to certain currently exemplary embodiments, the mutant BSF larvae of the present invention are produced by inserting a mutation within the Sema1a gene using the CRISPR/Cas system, a CRISPR/Cas homologous and CRISPR/Cas modified systems.

Clustered regularly interspaced short palindromic repeats (CRISPR)/Cas systems are known in the art and can be engineered for directed genome editing. Cas genes encode RNA-guided DNA endonuclease enzymes capable of introducing a double strand break in a double helical nucleic acid sequence. The Cas enzyme can be directed to make the double stranded break at a target site within a gene using the single guide RNA (sgRNA) and tracer cellular machinery.

The CRISPR/Cas system for genome editing contains two distinct components: a gRNA (guide RNA) and an endonuclease e.g., Cas9.

The gRNA is typically a 20-nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Comparable with other genome editing nucleases, Zinc-finger nucleases (ZFNs) and transcription activator-like effector nucleases (TALENs), the double-stranded brakes produced by CRISPR/Cas can undergo homologous recombination or nonhomologous end-joining (NHEJ).

The Cas9 nuclease has two functional domains: RuvC and HNH, each cutting a different DNA strand. When both domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present bi-allelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or homology directed repair (HDR) depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are number of publicly available tools to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools:Cas-OFFinder, the CasFinder:Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids.

Meltzer et al. (Meltzer H et al., 2019. Nat Commun 10:2113. doi.org/10.1038/s41467-019-10140-0) characterized tissue-specific (ts) CRISPR within the complex neuronal system of the Drosophila mushroom body. The generation of a library of gRNA-expressing plasmids and fly lines using optimized tools, and application of the library in a large-scale in vivo screen is described.

According to certain embodiments, reducing the expression and/or activity of the Sema1a protein is achieved by a method other than silencing the expression of the Sema1a gene using an RNA inhibiting molecule.

According to certain embodiments, reducing the expression and/or activity of the Sema1a protein is achieved by silencing the expression of the Sema1a gene using an RNA inhibiting molecule.

According to certain exemplary embodiments, the at least one mutation within the at least one allele is a deletion mutation. According to further exemplary embodiments, the at least one deletion comprises an exon or a part thereof.

According to certain exemplary embodiments, the Sema1a^{mut} allele comprises the nucleic acid sequence set forth in SEQ ID NO:3.

Any method as is known in the art for identifying BSF larvae or BSF flies comprising a mutated Sema1a encoding gene can be used according to the teachings of the present invention. According to certain currently exemplary embodiments, the Sema1a^{mut} allele is identified using a primer pair comprising SEQ ID NO:4 (GAGGAGGCCAACTAACAGTTCC) and SEQ ID NO:5 (TGGGCCCAATTCCTTATGGAG) amplifying a segment of about 400 bp.

According to certain embodiments, the at least one mutation in the Sema1a^{mut} allele is a deletion within the coding sequence of the Sema1a gene (SEQ ID NO:2).

According to certain embodiments, the deletion mutation leads to a premature stop codon. Various types of mutations can lead to a premature stop codon, including point mutations and frameshift mutations. The presence of the new stop codon results in the production of a shortened protein. According to embodiments of the invention the shorten protein has reduced or null function.

According to further certain exemplary embodiments, the at least one mutation is a deletion of the nucleotide A (Adenine) at position 80 of the nucleic acid sequence set forth in SEQ ID NO:2. According to these embodiments, the Sema1a^{mut} allele comprises the nucleic acid sequence set forth in SEQ ID NO:8.

According to yet additional certain exemplary embodiments, the at least one mutation is a deletion of the 10 nucleotides (CCTCGGAAAT) at position 72 to position 81 of the nucleic acid sequence set forth in SEQ ID NO:2. According to these embodiments, the Sema1a^{mut} allele comprises the nucleic acid sequence set forth in SEQ ID NO: 9.

According to some embodiments, the genetically modified BSFL is heterozygous to the Sema1a^{mut} allele.

According to some embodiments, the genetically modified BSFL is homozygous to the Sema1a^{mut} allele.

According to certain exemplary embodiments, the mutant Sema1a gene of the present invention is expressed throughout the BSFL tissues (i.e., the expression is not tissue-depended).

According to certain embodiments, the genetically modified BSFL comprise at least about 10%, at least about 15%, at least about 20%, at least about 25%, at least about 30%, at least about 35%, at least about 40%, at least about 45%, at least about 50%, at least about 55%, or more w/w protein out of the total weight of said BSFL on a dry weight basis.

According to certain exemplary embodiments, the genetically modified BSFL comprise from about 25% to about 50% w/w protein out of the total weight of said BSFL on a dry weight basis. According to certain further exemplary embodiments, the genetically modified BSFL comprise from about 30% to about 45% w/w protein out of the total weight of said BSFL on a dry weight basis.

According to certain embodiments, the genetically modified BSFL comprise at least about 10%, at least about 15%, at least about 20%, at least about 25%, at least about 30%, at least about 35%, at least about 40%, at least about 45%, at least about 50% fat w/w out of the total weight of said BSFL on a dry weight basis.

According to certain exemplary embodiments, the genetically modified BSFL comprise from about 20% to about 50% w/w fat out of the total weight of said BSFL on a dry weight basis. According to certain further exemplary embodiments, the genetically modified BSFL comprise from about 25% to about 40% w/w fat out of the total weight of said BSFL on a dry weight basis.

According to certain aspect, the present invention provides at least one fraction derived from a plurality of the genetically modified BSFL of the invention or a composition comprising same.

According to certain embodiments, the at least one fraction is selected from the group consisting of protein fraction, fat fraction, vitamin and mineral-containing aqueous fraction, chitin fraction, and any combination thereof.

Methods for extracting proteins, oil, chitin, or vitamin and mineral-containing aqueous fraction from BSFL are known in the art.

According to certain additional aspects, the present invention provides an edible composition comprising a plurality of the genetically modified BSFL of the invention and/or parts thereof.

According to certain embodiments, the edible composition further comprising at least one food-grade excipient or carrier.

According to certain embodiments, the edible composition comprises at least one additional nutritional component selected from the group consisting of at least one food-grade protein and/or amino acids, at least one food grade carbohydrate, at least one food grade fatty acid and any combination thereof.

According to certain embodiments, the edible composition comprises the genetically modified BSFL or parts thereof is in a form selected from the group consisting of a living form, a dried form, and a combination thereof.

According to certain embodiments, the dried genetically modified BSFL is in a form selected from the group consisting of whole larva and a larva meal.

According to certain embodiments, the dried genetically modified BSFL is defatted.

According to certain embodiments, the edible composition is for feeding a non-human animal. According to certain embodiments, the non-human animal is selected from the group consisting of a land animal and an aquatic animal. According to certain embodiments, the aquatic animal is selected from the group consisting of fish and crustacean. According to certain embodiments, the land animal is selected from the group consisting of avian, reptile, and mammal farm animal. According to certain embodiments, the animal is an insect. According to certain embodiments, the edible composition is a food for humans. According to these embodiments, the plurality of genetically modified BSFL is in a dried form.

According to further aspects, the present invention provides a method for producing a high-weight BSFL, the method comprising reducing the expression and/or activity of Sema1a protein within at least one cell of the BSFL. According to certain embodiments, the method results is a BSFL having higher weight compared to the weight of a corresponding BSFL having unmodified expression and/or activity of Sema1a protein.

According to certain embodiments, the method comprises introducing at least one mutation in at least one wild type allele of Sema1a to form Sema1a1^{mut} allele, wherein the at least one mutation confers a loss of function, a reduced function, or an abnormal function of the encoded Sema1a protein.

The wild type Sema1a protein, and the gene encoding same, and methods of generating the at least one mutation are as described hereinabove.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Generation of Sema1a^{mut}

### Mutagenesis of BSF Sema1a-I

Based on BLAST sequence identity (based on the nucleic acid sequence set forth in NCBI Reference Sequence: NC_051851.1), two sgRNA targeting sites named gRNA1 (or sgC, CCAGAATTACATCCGCACCA, SEQ ID NO:10), and gRNA2 (or sgB, TCAGACAATGAATTATATTC, SEQ ID NO:11) were identified. All gRNA templates described in this work used chemically synthesized guides RNA that were generated by Integrated DNA Technologies Inc. (IDT, Coralville, IA, USA). Cas9 protein was purchased from IDT as well. Fertilized eggs were collected at the time of laying and incubated at 30° C. Cas9 protein with the gRNA1 and gRNA2 molecules were microinjected into eggs. Injected eggs were incubated in a humidified chamber at 30° C. for 3-4 days until hatching. Hatched larvae were reared on chicken feed with 19% protein at 28° C. To identify somatic mutations, first instar larvae were selected for genomic DNA preparation. Fragments covering the two targeting sites were amplified with the following primers gRNA1 F (SEQ ID NO:4, GAGGAGGCCAACTAACAGTTCC) and gRNA2 R (SEQ ID NO:5, TGGGCCCAATTCCTTATGGAG). The amplified fragments were sequenced on the Sanger platform. Wild type alleles were amplified using the pair of primers gRNA1 F (SEQ ID NO:4) and gRNA1 R (SEQ ID NO:6, GCCTCAAGGGAGTAGTTGTTTGC) or the pair of primers gRNA2 F (SEQ ID NO:7, CTGGCTGTGCGCTCATATCTAG) and SEQ ID NO:5.

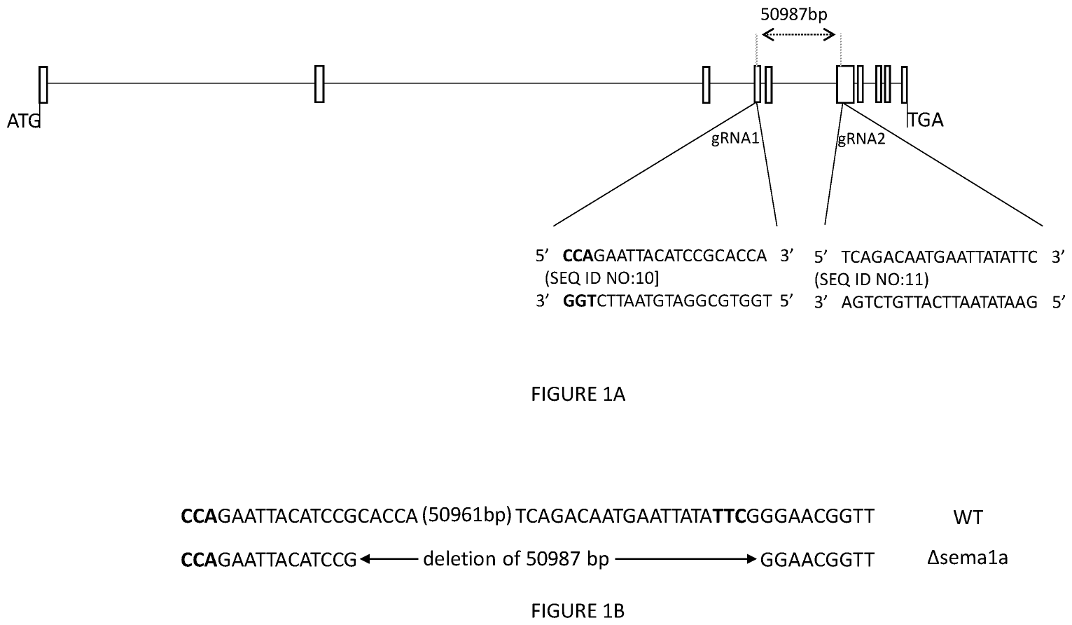

Using the CRISPR/Cas9 system, a BSF line comprising a deletion mutation in the Sema1a gene was generated (FIG. 1). The mutation was a deletion of 50987 bps from position 419,698 to position 470,684 on the genomic sequence encoding Sema1a1 which is present between position 159,092,514 and position 159,592,196 of the nucleic acid sequence of NCBI Reference Sequence NC_051851.1. This Sema1a^{mut} allele comprises SEQ ID NO:3.

### Mutagenesis of BSF Sema1a-II

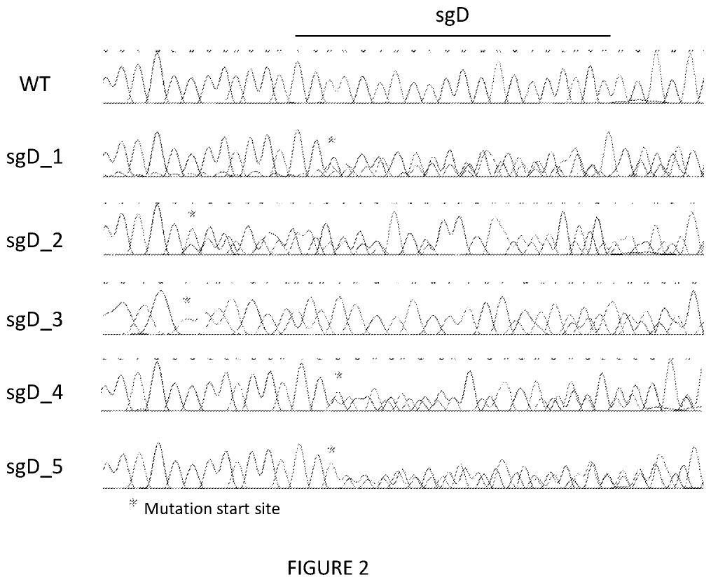

Two additional sgRNA targeting sites designated sgA (GCCAGGCACTTAAATTTCCG, SEQ ID and sgD NO: 12) (TGTGGACTCGGACTACTTGA, SEQ ID NO:13) were each injected to fertilized eggs along with Cas9 protein as described hereinabove. Injected eggs (embryos) were allowed to develop into adults and inter-mated. F1 larvae of eggs injected with sgD and were grown under optimal conditions, and individual larvae exhibiting delayed blackening phenotype were collected and genotyped. In all larvae showing the mutant phenotype of increased weight and a later pupation (Table 1), a mutation at the targeted area was observed (FIG. 2). These results show that various mutations in the Sema1a encoding gene may lead to the desired outcome of increased larval weight at the end of the 5^{th} larval stage (before pupation).

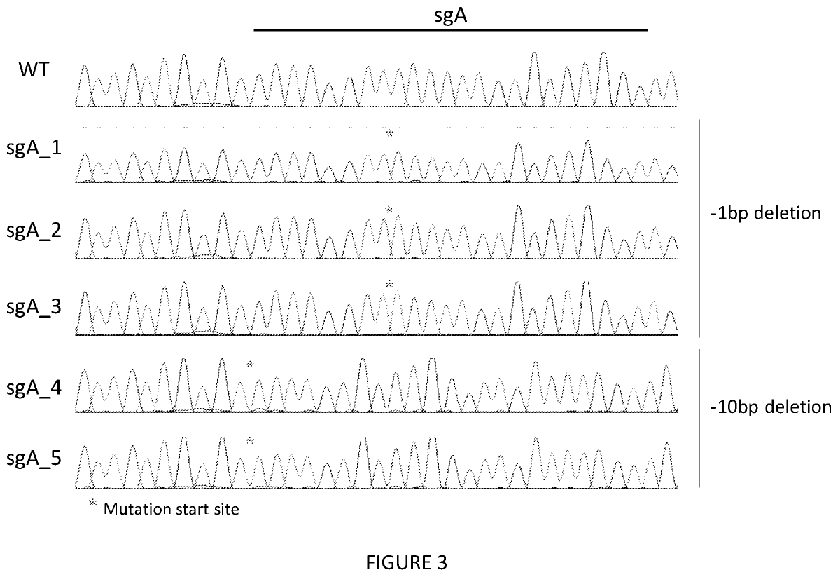

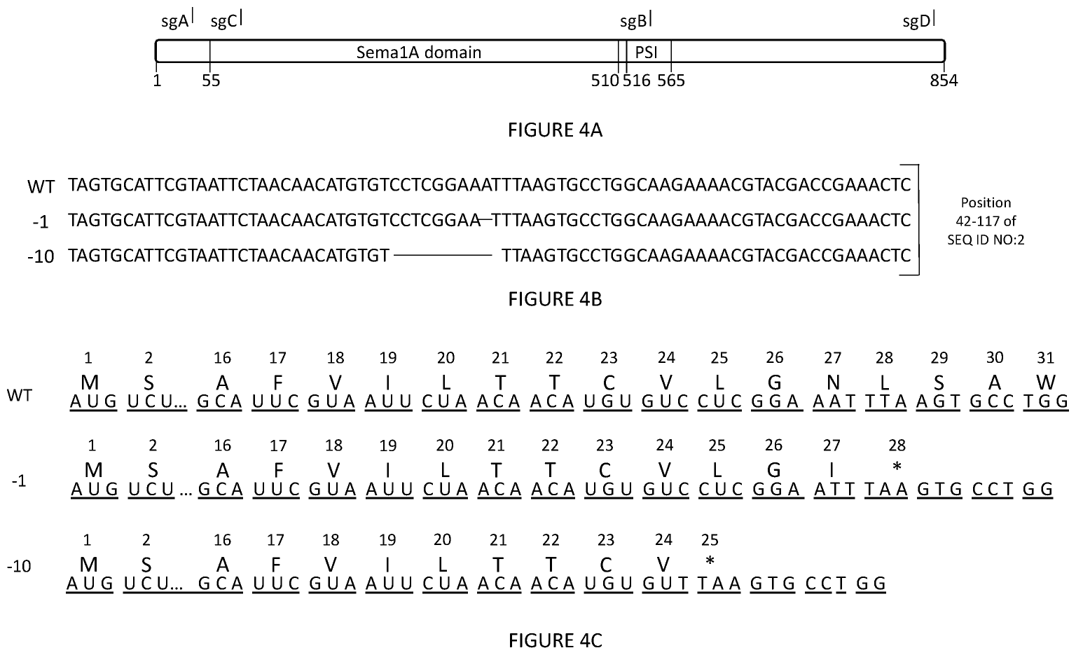

Offspring larvae of eggs injected with sgA (further to F1) showed two specific deletion mutations: a point deletion of the nucleotide A (adenine) at position 80 of SEQ ID NO: 2 (forming Sema1a^{mut} allele comprises SEQ ID NO:8) and a deletion of 10 nucleotides between positions 72-81 of SEQ ID NO:2 (forming Sema1a^{mut} allele comprises SEQ ID NO:9, FIG. 3 and FIG. 4). Both mutations are frameshift mutation that causes a premature stop codon, resulting in a non-active Sema1a protein, leading the phenotype of increased weight of the mutant larvae (Table 2).

TABLE 1
Weight of wild-type larvae and larvae
hatched from Cs9-sgD injected eggs

| | Final Weight | Delay in Blackening |
|---|---|---|
| Sample | (mg) | (Days after wild type) |
| WT | 255 | 0 |
| sgD1 | 366 | 4 |
| sgD2 | 337 | 4 |
| sgD3 | 335 | 4 |
| sgD4 | 330 | 4 |
| sgD5 | 328 | 4 |

TABLE 2
Weight of wild-type larvae and larvae
hatched from Cs9-sgA injected eggs

| | Final Weight | Delay in Blackening |
|---|---|---|
| Sample | (mg) | (Days after wild type) |
| WT | 255 | 0 |
| sgA1 | 451 | 4 |
| sgA2 | 385 | 4 |
| sgA3 | 382 | 4 |
| sgA4 | 385 | 4 |
| sgA5 | 388 | 4 |

### Example 2: Characteristics of the BSF Larvae Comprising the Sema1a^{mut} Allele

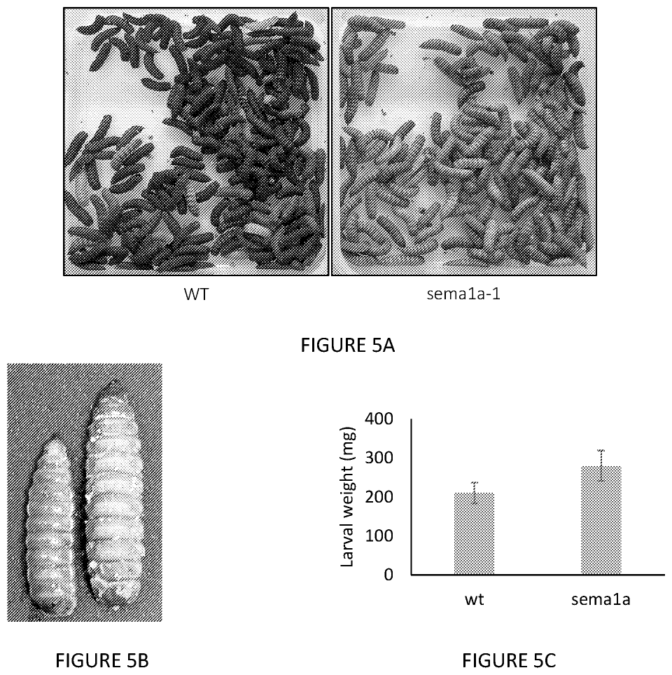

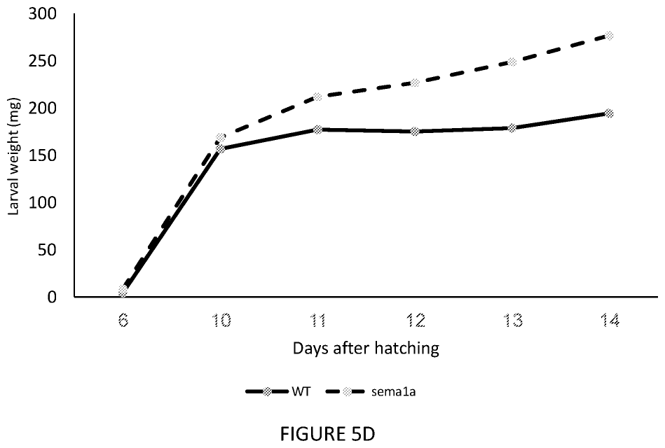

Larvae hatched from eggs laid by flies mutated as described in Example 1 hereinabove were late to metamorphose into the pupal stage. FIG. 5 shows wilt type and mutant larvae comprising the Sema1a^{mut} comprising SEQ ID NO:3. At 16 days after hatching, control larvae have turned black, indicating reach of the pre-pupation stage (FIG. 5A, left panel), compared to the mutant larvae, which maintained their bright color and continued to grow (FIG. 5A, right panel). Representative picture of CRISPR mutant larva of the Sema1a gene comprising SEQ ID NO:3 and WT control larva at day 14 after egg laying is shown in FIG. 5B. The average weight of the mutated larvae was significantly higher compared to the weight of the wild type larvae (average of 280 mg vs. 210 mg, respectively, FIG. 5C). The growth pattern of the mutated larvae shows higher weight throughout day 10 to day 14 (FIG. 5D).

Flies matured from the mutated larvae were also larger (FIG. 5E) and had a significant higher average weight compared to flies matured from wild type larvae (91.5 mg vs. 56.1 mg, respectively, FIG. 5F).

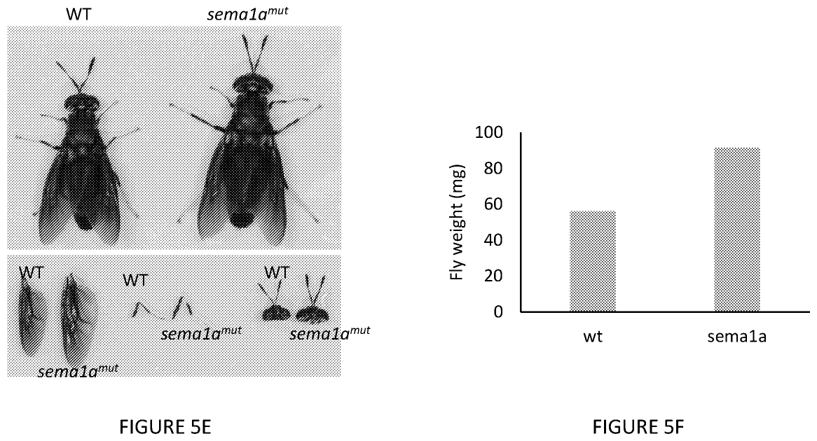

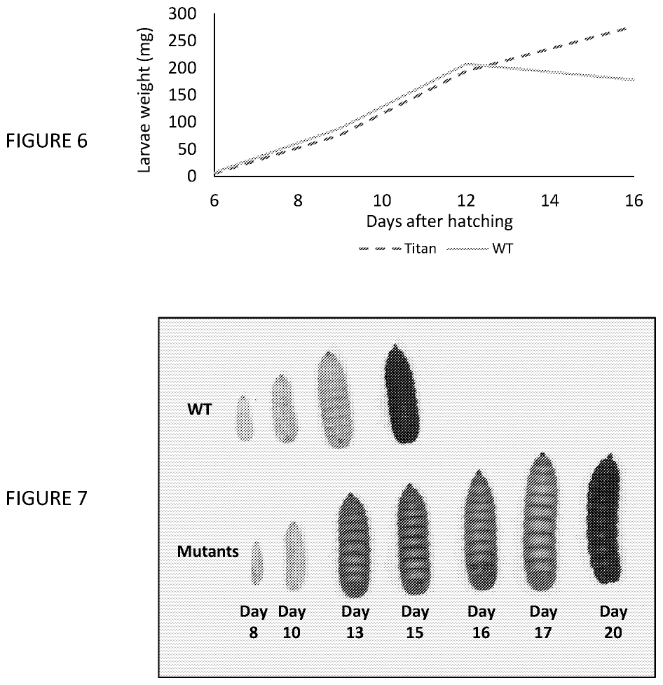

FIG. 6 shows time-course data for weight gain in Sema1a^{mut} comprising SEQ ID NO: 9 and WT larvae. The body mass of both groups increased over time, with mutants showing a growth pattern similar to or slightly slower than that of the control larvae. Significantly, while the control larvae ceased feeding and growing at day 12 After hatching from the egg (16 days after egg laying), mutant larvae exhibited continued growth until day 16 after hatching (20 days after egg laying). This prolonged growth phase contributed to a significant elevation in the final body mass of the mutant larvae compared to their wild-type counterparts. FIG. 7 shows that while both the WT and mutant larvae reached the stage of blackening, the mutant larvae reached this stage at a later day after egg laying having higher weight.

In summary, the present invention shows for the first time BSF larvae comprising a mutant Sema1a gene, wherein expression of the mutated gene is not directed to a certain time or organ, such that the mutated gene is expresses constitutively and throughout the larval body. The BSFL carrying the mutant allele showed a delayed maturity and an increase in the body weight. Furthermore, unexpectedly, the mutant larvae were capable to metamorphose and reach the adult fly stage as high weight, fertile flies.

### Example 3: Measurements of Feed Conversion Ratio (FCR)

200 mutant and 200 wild type larvae were grown separately on an equal amount of food. At the end of the experimental trial on day 12, all larvae were weighed. FCR was calculated according to the following equation:

$$FCR = \frac{substrare consumption ( g wet weight )}{\begin{matrix}post trial total larvae mass ( g wet weight ) - \\ initial total larvae mass ( g wet weight )\end{matrix}}$$

An improvement of about 20% in FCR was observed for the genetically modified larvae (FCR value of 3.305 compared to a value of 4.160 for the WT larvae).

### Example 4: Larvae Nutritional Value

The nutritional value of the mutant larvae was compared to that of wild type larvae.

The proximate composition of wild type and sema1a mutant larvae at the same developmental stage was analyzed at Milouda & Migal laboratories, Israel, using established AOAC International Official Methods Program as follows: Moisture analysis Based on AOAC 950.46. Crude protein analysis based on AOAC976.05,950.36,991.20 and 986.25. Fat by hydrolysis analysis based on Nestle LI 00.527-1. For Ash analysis based on AOAC 923.03.

The results are presented in Table 3. The observed similar moisture value indicates that the mutant higher weight is not due to excess water. A similar percentage of crude protein and crude fat indicates that the mutant protein and fat composition is not altered compared to the WT strain, yet, due to the higher total weight of the mutant larvae, these larvae provide for higher weight of protein and fat.

TABLE 3
Larvae nutritional value

| | |
|---|---|
| Experiment I | Experiment II |
| Sema1a^{mut} | Sema1a^{mut} |
| comprising SEQ | comprising SEQ |
| ID NO: 3 | ID NO: 9 |

| | WT | Mutant | WT | Mutant |
|---|---|---|---|---|
| Moisture | 67.2 | 65.9 | 66.54 | 66.81 |
| (g/100 g) | | | | |
| Protein (%) | 48.8 | 43.3 | 48 | 48 |
| Fat (%) | 36.8 | 35.8 | 28 | 32 |
| Ash (%) | 6.3 | 6.1 | 6.2 | 7.5 |

### Example 5: Life Cycle of BSF Comprising Sema1a^{mut} Allele

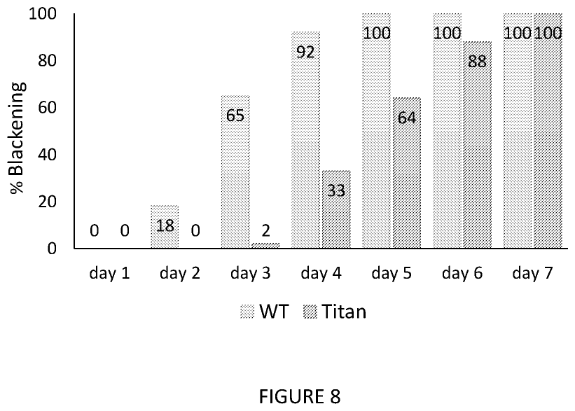

To further characterize the effect of the mutation of BSF life cycle, 100 wild-type (WT) larvae and 100 mutant larvae (“Titan” larvae comprising Sema1a^{mut} allele comprising SEQ ID NO:9) were monitored from late larva stage to per-pupa stage. The black to white ratio of population was documented daily. 100% of the individual larvae from both the wild-type and mutant groups underwent metamorphosis into the pupa stage (FIG. 8). Furthermore, fly emergence from the mutant pupae was not negatively affected and was similar to that of wild type pupae: out of 100 mutant pupae, 96 flies emerged, and out of 100 wild type pupae, 94 flies emerged.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-53. (canceled)

54. A genetically modified black soldier fly larva (BSFL) having reduced expression and/or activity of Semaphorin1a (Sema1a) protein or a homolog thereof, wherein the weight at the end of the 5th larval stage of the genetically modified larva is higher than the weight of a corresponding, unmodified BSFL grown under similar conditions and being at the same larval stage.

55. The genetically modified BSFL of claim 54, wherein said genetically modified BSFL is capable to metamorphose into pupae.

56. The genetically modified BSFL of claim 54, wherein said genetically modified BSFL is capable to metamorphose into adult fly, and wherein the adult fly has an increased weight compared to the weight of an adult fly metamorphosed from the corresponding unmodified BSFL.

57. The genetically modified BSFL of claim 54, wherein said genetically modified BSFL comprises within its genome at least one mutant allele of the Sema1a gene or of the homolog thereof, wherein the mutant allele comprises at least one deletion mutation.

58. The genetically modified BSFL of claim 57, wherein the deletion is within the genomic sequence encoding the Sema1a protein, wherein said genomic sequence is at least 85% identical to the nucleic acid sequence set forth between position 159,092,514 and position 159,592,196 within the nucleic acid sequence set forth in NCBI Reference Sequence NC_051851.1.

59. The genetically modified BSFL of claim 58, wherein the deletion is of 50987 nucleotides from position 159,121,513 to position 159,172,499 on the nucleic acid sequence set forth in NCBI Reference Sequence: NC_051851.1.

60. The genetically modified BSFL of claim 59, wherein the mutant allele of Sema1a comprises the nucleic acid sequence set forth in SEQ ID NO:3.

61. The genetically modified BSFL of claim 57, wherein the deletion is within the coding sequence of the Sema1a protein, wherein said coding sequence is at least 85% identical to SEQ ID NO:2, and wherein the deletion is selected from the group consisting of (i) a deletion of the nucleotide A at position 80 of the nucleic acid sequence set forth in SEQ ID NO:2 and (ii) a deletion of 10 nucleotides between position 72 and position 81 of the nucleic acid sequence set forth in SEQ ID NO:2.

62. The genetically modified BSFL of claim 54, wherein the weight of said genetically modified BSFL is at least 10%, at least 20%, at least 30%, at least 40%, at least 50% higher compared to the weight of the corresponding unmodified BSFL.

63. The genetically modified BSFL of claim 54, wherein said genetically modified BSFL comprise at least 10%, at least 20%, at least 30%, at least 40%, or at least 50% w/w fat out of the total weight of said BSFL based on a dry weight.

64. The genetically modified BSFL of claim 54, wherein said genetically modified BSFL comprise at least 10%, at least 20%, at least 30%, at least 40%, or at least 50% w/w protein out of the total weight of said BSFL based on a dry weight.

65. An edible composition comprising a plurality of the genetically modified BSFL of claim 54.

66. The edible composition of claim 65, wherein the genetically modified BSFL is in a dried form and wherein said edible composition comprises said genetically modified BSFL in a form selected from the group consisting of whole larva and a larva meal.

67. The edible composition of claim 66, wherein the dried genetically modified BSFL is defatted.

68. A method for producing a high-weight BSFL, the method comprising reducing the expression and/or activity of Sema1a protein within at least one cell of the BSFL.

69. The method of claim 68 wherein said method comprises generating at least one mutation in at least one wild type allele of Sema1a or a homolog thereof to form Sema1a1 mutant allele, wherein the at least one mutation confers a loss of function, a reduced function, or an abnormal function of the encoded Sema1a protein.

70. A high-weight genetically modified BSFL produced by the method of claim 69.

71. A composition comprising at least one fraction derived from the genetically modified BSFL of claim 54.

72. The composition of claim 71, wherein the at least one fraction is selected from the group consisting of protein fraction, fat fraction, chitin fraction, vitamin and mineral-containing aqueous fraction and any combination thereof.

73. A method of converting an organic waste to biomass, the method comprises providing a plurality of the genetically modified BSFL of claim 54 with organic waste as the sole nutrient source.
